(19) United States
(12) Patent Application Publication
(10) Pub. No.: US 2007/0036874 A1
(43) Pub. Date: Feb. 15, 2007

(54) Compositions and methods for controlling glucose and lipid uptake from foods

(76) Inventor: Litao Zhong, San Diego, CA (US)

Correspondence Address: MORRISON & FOERSTER LLP, 12531 HIGH BLUFF DRIVE, SUITE 100, SAN DIEGO, CA, 92130-2040, US

(21) Appl. No.: 11/499,603

(22) Filed: Aug. 4, 2006

Related U.S. Application Data
(60) Provisional application No. 60/705,961, filed on Aug. 5, 2005.

(51) Int. Cl. A61K 36/82; A61K 31/445; A61K 31/7048; A61K 31/353

(52) U.S. Cl. 424/729; 424/774; 514/327; 514/27; 514/456

(57) ABSTRACT

The invention relates to compositions comprising three inhibitors, one pancreatic lipase inhibitor, an alpha glucosidase inhibitor, and a sodium dependent glucose transporter inhibitor. The invention also provides methods of using the compositions for controlling glucose and lipid uptake.

### CROSS-REFERENCE TO RELATED APPLICATIONS

This application claims benefit of U.S. provisional patent application Ser. No. 60/705,961, filed Aug. 5, 2005, which is incorporated herein by reference in its entirety.

### FIELD OF THE INVENTION

This invention relates to methods and compositions for controlling glucose and lipid uptake by administering a combination of three inhibitors, one lipase inhibitor (fermented tea extract) and one alpha glucosidase inhibitor (Mulberry leaf extract) and at least one sodium dependent glucose transporter inhibitor (epicatechin gallate from green tea extract).

### BACKGROUND OF THE INVENTION

Glucose and lipid metabolism play important roles in the development of diabetes and obesity, and restricted glucose and lipid uptake is an effective therapeutic means for diabetes and obesity. For example, Pereira et al reported that after four months of restricted glucose uptake, insulin resistance, serum triglycerides, C-reactive proteins and blood pressure were significantly improved in tested human subjects (1). It was also reported that people on a low-carbohydrate diet has lost significantly more weight than subjects on the conventional low-fat diet at 3 months and 6 months (2). Excessive lipids, or dietary fat uptake has been widely accepted as one of the main causes of obesity (21).

A substantial portion of glucose uptake in daily life comes from starch. After ingestion, starches are first broken down into complex sugars by amylase in saliva and in intestine. The complex sugars are then turned into glucose by glucosidases. Finally, the glucose crosses the lining of intestine, mainly through a sodium dependent glucose transporter and enters into blood stream (3). The metabolism of starch is described below:

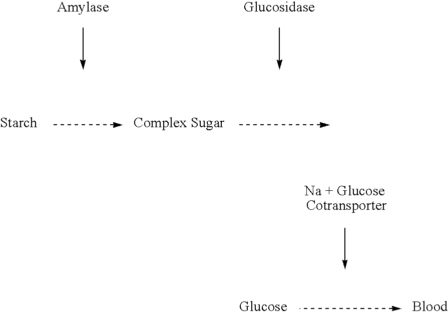

Inhibitors of alpha amylase, a major amylase in the body, were found in white kidney bean extract and in wheat extract as well as in tea extract (4, 5, 22). Human as well as animal studies indicated effectiveness of these extracts in decreasing starch metabolism (6, 7). Their usage in body weight management were speculated and discussed (8, 9). Some of the commercially available amylase inhibitors from white kidney bean extracts were named as a “starch blocker” by their marketers and were widely sold as dietary supplement for body weight management. However, starch metabolism prevention by these extracts has not been satisfactory, and certainly far from complete. In fact, published studies showed that the “starch blockers” were ineffective in body weight management in both animals and in human (10).

Another major source of glucose uptake is from sucrose consumed every day. Sucrose, also called as cane sugar, beet sugar, maple sugar and even “table sugar”, appears in most soft drinks and in all sorts of foods such as desserts. It is a disaccharide, consisting of one unit of glucose and one unit of fructose. After ingestion, sucrose is hydrolyzed into glucose and fructose by glucosidase in the small intestine.

Triglyceride, or neutral lipid, is the major form of daily food fat. However, triglyceride cannot cross the intestinal mucosa before broken down by pancreatic lipase into 2-monoglyceride and two free fatty acids. Because of this, pancreatic lipase has been regarded as a target for obesity management. At least one drug, Orlistat (Xenical), which is a pancreatic lipase inhibitor, has been developed to reduce the absorption of dietary fat. Clinical studies have demonstrated its efficacy in body weight management.

There remains a continuing need for new effective methods for controlling glucose and lipid uptake.

### BRIEF SUMMARY OF THE INVENTION

The present invention provides a composition comprising a pancreatic lipase inhibitor, an alpha glucosidase inhibitor, and a sodium dependent glucose transporter inhibitor. It is contemplated that the combination may produce at least an additive effect or a synergistic effect compared to each component individually.

In one embodiment, the pancreatic lipase inhibitor comprises fermented tea extract such as polymerized catechins. In another embodiment, the alpha glucosidase inhibitor comprises a mulberry leaf extract. In yet another embodiment, the sodium dependent glucose transporter inhibitor comprises epicatechin gallate. In some embodiments, said sodium dependent glucose transporter inhibitor comprises a green tea extract.

In some embodiments, the composition comprises a pancreatic lipase inhibitor provided by a fermented tea extract, an alpha glucosidase inhibitor provided by a mulberry leaf extract, and a sodium dependent glucose transporter inhibitor provided by a green tea extract.

In some embodiments, the composition further comprises a pharmaceutically acceptable carrier. Two or more inhibitors (i.e., pancreatic lipase inhibitor, alpha glucosidase inhibitor and sodium dependent glucose transporter inhibitor) may be in a co-formulation or in a separate formulation. In other embodiments, two or more inhibitors may be in tablets, capsules, powders or beverages. The composition may be included in food product or beverage.

The invention also provides a method for controlling lipid and glucose uptake in an individual, comprising administering to the individual an alpha glucosidase inhibitor, a sodium dependent glucose transporter inhibitor, and a pancreatic lipase inhibitor, whereby said three inhibitors in conjunction provide effective control of lipid and glucose uptake. The methods of the present invention may be used for treating or preventing diabetes (including type I and type II), cardiovascular diseases, obesity, or overweight. An example of a pancreatic lipase inhibitor is polymerized catechin from fermented tea.

In some embodiments, the three inhibitors (i.e., pancreatic lipase inhibitor, alpha glucosidase inhibitor and sodium dependent glucose transporter inhibitor) are administered simultaneously. In some embodiments, the three inhibitors are administered at different times.

In some embodiments, a pancreatic lipase inhibitor, an alpha glucosidase inhibitor, and a sodium dependent glucose transporter inhibitor are administered in a single formulation. The inhibitors may be administered orally before a meal, or with a meal.

The invention also provides a kit for used in any of the methods described herein comprising an alpha glucosidase inhibitor, a sodium dependent glucose transporter inhibitor, and a pancreatic lipase inhibitor. The kit may further comprise instructions for any of the methods described herein, such as instructions for administering the inhibitors. The pancreatic lipase inhibitor, alpha glucosidase inhibitor and sodium dependent glucose transporter inhibitors may be packaged together, but may or may not be in the same container.

In another aspect, the invention provides methods for inducing carbohydrate malabsorption, comprising administering to an individual a composition comprising a pancreatic lipase inhibitor, an alpha glucosidase inhibitor, and a sodium dependent glucose transporter inhibitor, thereby inducing carbohydrate malabsorption. In one embodiment, the pancreatic lipase inhibitor comprises theaflavin or a black tea extract; the alpha glucosidase inhibitor comprises a mulberry leaf extract; and the sodium dependent glucose transporter inhibitor comprises a green tea extract.

The invention also provides for the use of a composition comprising a pancreatic lipase inhibitor, alpha glucosidase inhibitor and sodium dependent glucose transporter inhibitor for controlling glucose and lipid uptake in an individual. Furthermore, the invention provides the use of a composition comprising a pancreatic lipase inhibitor, alpha glucosidase inhibitor and sodium dependent glucose transporter inhibitor for inducing carbohydrate malabsorption in an individual. In particular embodiments, the individual has diabetes or is at risk of diabetes. In other embodiments, the individual is obese or is at risk of obesity.

### BRIEF DESCRIPTION OF THE FIGURES

FIG. 1 shows the mean±sem of breath H₂ concentrations for 20 volunteers ingesting a meal of rice (50 g carbohydrate), butter (10 g), 0.2 g ¹³C-triolein, with a fermented tea extract, green tea extract and mulberry extract combination preparation (solid circles) or a placebo solution (open squares), both of which contained 10 g of sucrose.

### DETAILED DESCRIPTION OF THE INVENTION

The present invention provides compositions and methods for controlling lipid and glucose uptake into the body of an individual.

### I. Definitions

Unless defined otherwise, all technical and scientific terms used herein have the same meaning as is commonly understood by one of ordinary skill in the art to which this invention belongs. All patents, patent applications (published or unpublished), and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

As used herein, “treatment” is an approach for obtaining beneficial or desired clinical results. For purposes of this invention, beneficial or desired clinical results include, but are not limited to, one or more of the following: improvement or alleviation of any aspect of controlling glucose and lipid uptake, maintaining healthy blood glucose and lipid level, and maintaining body weight.

An “effective amount” is an amount sufficient to effect beneficial or desired clinical results including controlling glucose and lipid uptake. An effective amount, in the context of this invention, may also be amounts of using two or more inhibitors described herein such that synergy is achieved. An “effective amount” of two or more inhibitors described herein can result in a synergistic effect as compared to administering each inhibitor alone.

An “individual” is a mammal, more preferably a human. Mammals also include, but are not limited to, farm animals, sport animals, pets, primates, horses, cows, dogs, cats, mice and rats.

As used herein, administration “in conjunction” includes simultaneous administration and/or administration at different times. Administration in conjunction also encompasses administration as a co-formulation or administration as separate compositions. As used herein, administration in conjunction is meant to encompass any circumstance wherein at least two inhibitors described herein are administered to an individual, which can occur simultaneously and/or separately. As further discussed herein, it is understood that two or more inhibitors can be administered at different dosing frequencies or intervals. It is understood that two or more inhibitors can be administered using the same route of administration or different routes of administration.

### II. Compositions

The present invention provides a composition comprising a pancreatic lipase inhibitor, an alpha glucosidase inhibitor, and a sodium dependent glucose transporter inhibitor.

Alpha glucosidase is the dominant glucosidase in the body. The enzyme hydrolyses disaccharides into monosaccharide such as glucose. Alpha glucosidase inhibitors has been shown to be an effective means in decreasing glucose uptake and thus offering potential therapeutics to diabetic patients (11). Some alpha glucosidase inhibitors were successfully developed into prescription drugs, such as Acarbose and Miglitol, two synthetic drugs widely used by diabetic patients (12). Because of its mechanism in glucose metabolism, glucosidase inhibitors have been expected to be useful in body weight management. A clinical study showed that a high dose of Acarbose possesses relapse-reduction effects after weight reduction in severely obese patients (13). There were also reports showing significant weight loss in a type 2 diabetic patient after using Acarbose (13).

Mulberry (Morus alba) leaf has been used in Chinese traditional medicine for hundreds of years as a “cooling” herb to remove excessive heats and toxins from the body. In recent years, however, more and more attention has been put on its anti-diabetic properties. Alkaloids and N-containing sugars isolated from Mulberry leafs were found as potent inhibitors of alpha glucosidase. One report also suggested that ecdysterone found in Mulberry turned glucose into glycan. Both animal and human clinical studies of a proprietary extract (SUCRALITE™) of Mulberry leaf extract demonstrated its efficacy in decreasing postprandial blood glucose in normal and diabetic patients. It was also found that SUCRALITE™ is capable of relieving some symptoms of diabetes. The efficacy of the extract was shown as similar to that of synthetic drug Acarbose. Animal toxicity studies demonstrate that SUCRALITE™ Mulberry leaf extract is safe. One advantage of alpha glucosidase inhibitors, such as Mulberry extract, over the amylase inhibitor, such as phaseolamin, is that they diminish glucose production not only from starch, but also from other sources, such as sucrose, the table sugar.

Sodium dependent Glucose transporter is the main means through which glucose enter into blood from intestine (3, 14). One in vitro study showed that 90% of glucose enters blood stream through this transporter (3). Epicatechin gallate, a polyphenol isolated from green tea, was found to be a potent inhibitor of sodium dependent glucose transporter via a competitive mechanism (14). An in vitro study demonstrated that up to 50% of the glucose uptake through incubated intestinal membranes was inhibited by Epicatechin gallate. Based on these discoveries, it is expected that Epicatechin gallate has the potential to reduce the glucose uptake from all sources, including, starch and sugar.

Ingredients isolated from tea extracts, such as theaflavin and epigallocatechin gallate, have been found to be effective inhibitors for lipase (17, 18, 19, and 20). It was also demonstrated that tea catechins decrease the solubility of cholesterol in micelles and reduce intestinal cholesterol absorption (23). Animal studies showed that both green tea and black tea extract increased fecal excretion of fat (24, 25).

In one embodiment, the compositions further comprise a pharmaceutically acceptable excipient or carrier. In some embodiments, the composition is for use in any of the methods described herein (such as methods for treating diabetes and/or obesity). The inhibitors of the composition may be present in a single formulation or present as separate formulations. Accordingly, in some embodiments, the inhibitors are present in the same formulation. In other embodiments, each inhibitor is present in a separate formulation.

It is understood that the composition can comprise more than one inhibitor for each of the pancreatic lipase inhibitor, the alpha Glucosidase inhibitor, and the sodium dependent glucose transporter inhibitor. The inhibitors may be provided by herbal extract, such as mulberry leaf extract, green tea extract and fermented tea extract. One extract may contain more than one type of inhibitors.

The composition used in the present invention can further comprise pharmaceutically acceptable carriers, excipients, or stabilizers (Remington: The Science and Practice of Pharmacy 20th Ed. (2000) Lippincott Williams and Wilkins, Ed. K. E. Hoover), in the form of lyophilized formulations or aqueous solutions. Acceptable carriers, excipients, or stabilizers are nontoxic to recipients at the dosages and concentrations, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); polypeptides of low molecular weight (less than about 10 residues); proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Pharmaceutically acceptable excipients are further described herein.

### III. Exemplary embodiments

The combination of three herbal extracts, i.e., fermented tea extract (containing pancreatic lipase inhibitors, such as polymerized catechin), Mulberry leaf extract (containing alpha glucosidase inhibitors, such as 1-deoxynojirimycin) and epicatechin gallate (from green tea extract, to block the glucose transporter), may act synergistically and have a strong effect in diminishing lipid and glucose uptake into the body, and thus have strong effects in maintaining healthy blood glucose and lipid level and body weight.

A. Description of products

1. Mulberry Leaf Extract

Mulberry leafs, dry or fresh, are extracted with water/alcohol solution. The solution is dried with vacuum to remove alcohol and a water precipitation is followed. The precipitates are removed by filtration or centrifuge. The supernatant is dried and re-dissolved in water. The solution is then loaded to a column chromatography, and the fraction having alpha Glucosidase inhibition activity is eluted and dried. The eluted material should have 50-100% (more specifically 80-90%) inhibition on alpha glucosidase, based on an in vitro alpha glycosidase inhibition assay (16).

Mulberry leaf extract is commercially available and is available for example, from NatureGen, Inc. (San Diego, Calif.).

2. Epicatechin Gallate (ECG)

ECG can be prepared from green tea leaf extract. Tea leafs are water extracted at 80° C. The solution is then extracted with ethyl acetate. The ethyl acetate fraction is dried and re-dissolved in alcohol and water solution. The solution is then loaded on column chromatography, and Epicatechin Gallate is eluted by alcohol solution wash. The compound should exert about 50% or more inhibition on sodium dependent glucose transporter based on a published assay (14).

Epicatechin Gallate is commercially available, and is available for example, from NatureGen (between 10-50% in purity).

3. Polymerized Catechins

Polymerized catechins are prepared from fermented catechins. Tea catechins are incubated with polyphenol oxidase in a reaction tank for 60 to 120 minutes. The mixture is then extracted with ethyl acetate and dried. In particular embodiments, the extracts have at least 10 USP units human pancreatic lipase inhibition per mg.

LIPOTAME™, an exemplary polymerized catechin commercially available from NatureGen, has 11.62 USP units/mg of lipase inhibition activity, compared to 80 USP units/mg of lipase inhibition activity found in ORLISTAT®.

B. Products Administration

The intended usage of the combinations is one to three times a day, before or with meal. Each serving comprises: Mulberry extract (50 mg to 1500 mg, more specifically 500 mg to 1000 mg); Epicatechin Gallate (10 mg to 1000 mg, more specifically 100 mg-300 mg); and fermented tea extract (10 mg to 500 mg, more specifically 100-300 mg).

C. Dosage Form

The combinations can be in tablets and/or capsules or softgels; powders; beverage; or food (such as pizza or pasta or bar, ingredients)

D. Examples of Use

1. Tablets and Capsules and Softgels:

In order to control blood glucose and lipid and/or body weight, a subject may take 2-4 tablets or capsules of softgel with water before each meal. Each capsule or tablet of softgels contains 100 mg fermented tea extract, 250 mg Mulberry extract and 200 mg epicatechin gallate with other inactive excipients.

2. Powders

In another embodiment, a subject may mix a spoonful of blended powder with water, and drink the mixture prior to a meal. The powder may contain 75 mg fermented tea extract, 500 mg Mulberry extract and 150 mg epicatechin gallate and other inactive excipients including flavor additives such as lemon, orange or banana.

3. Beverage

In other embodiments, a subject may drink a liquid preparation containing 150 mg fermented tea extract, 500 mg Mulberry extract and 150 mg epicatechin gallate with other inactive excipients including flavor additives such as lemon, orange or banana.

4. Low Glycemic Index Food, Such as Pasta, Bread, Pizza or Bar

Foods, including pasta, bread, pizza, bar and etc, can be made with the addition of the above three ingredients or any two of the ingredients. These foods will become low glycemic index food, because much less glucose is going to be produced and absorbed by the body in comparing with foods without the addition of these ingredients.

5. Pre-mixed Powders

All three ingredients (in the powder form) can be mixed or blended in a specific ratio. In one example, theaflavin, Mulberry extract and epicatechin gallate are mixed in a ratio of 75:500:75 to produce a premixed powder. Such premixed powder can be used to make capsules, tablets, beverages and/or used as food ingredients.

Although the foregoing invention has been described in some detail by way of illustration and example for purposes of clarity of understanding, the descriptions and examples should not be construed as limiting the scope of the invention.

### IV. Carbohydrate Malabsorption Study

A study utilizing measurements of breath H₂ was used to investigate the ability of a combination of fermented, green and mulberry tea leave extracts to induce malabsorption of carbohydrate in healthy volunteers.

Results in studies below indicate that with the carbohydrate-containing meal, the tea extract combination resulted in a highly significant increase in breath H₂ concentration indicating appreciable carbohydrate malabsorption. Comparison of this H₂ excretion with that previously reported following ingestion of the non-absorbable disaccharide lactulose suggested that the combination induced malabsorption of 25% of the carbohydrate. The combination did not cause any significant increase in symptoms.

A. Design

In a crossover design, healthy adult volunteers randomly ingested test meals with a placebo beverage or a preparation containing an extract of black (0.1 g), green (0.1 g) and mulberry (1.0 g) teas. The test meal contained 50 g of carbohydrate as white rice, 10 g of butter, and 0.2 g of ¹³C-triolein, and the beverages contained 10 g of sucrose. Breath H₂ concentrations were assessed hourly for 8 hours, and symptoms were rated on a linear scale.

Twenty healthy volunteers (ages 23 to 60, 10 females and 10 males fasted after their usual dinner until the following morning (approximately 8 am) when the experiments were performed at the Minneapolis Veterans Administration Medical Center. After collection of baseline breath samples for H₂ analysis, the subjects ingested a test meal of white rice and butter. The rice was boiled for 20 minutes, and then individual portions (176 g containing 50 g of carbohydrate) were frozen with 10 g of butter. Immediately prior to ingestion, the meals were warmed in a microwave oven, and 0.2 g of ¹³C-triolein (Cambridge Isotope Laboratories, Andover, Mass.) was thoroughly mixed into the meal. Five hundred ml of warm water and 10 g of sucrose were added to the tea or placebo preparations, which were well stirred.

The subjects were assigned randomly to drink either the tea or the placebo concurrently with the meal. Breath samples were then collected at hourly intervals for eight hours. At the end of each test period, subjects were asked to rate a variety of symptoms including nausea, bloating, abdominal discomfort, and rectal gas (as well as obfuscating symptoms) on a previously described linear scale that ranged from zero (none) to 4 (severe). (See e.g., Suarez et al., Nutritional supplements used in weight reduction programs increase intestinal gas in persons who malabsorb lactose. J Am Diet Assoc 101:1447-52 (2001)). In addition, loose bowel movements were noted. One week later the test was repeated with the subjects receiving the opposite preparation from that of the initial study.

1. Test Products

The active preparation, a proprietary product, contains a mixture of extracts of green tea (0.1 g), fermented (or black) tea (0.1 g), and mulberry (1.0 g) tea leaves. The control beverage contained trace quantities red dye #40 and caramel to provide a brown color similar to that of tea. (Both products were supplied by NatureGen, Inc., San Diego, Calif.). The taste of the two test materials differed, and subjects were aware of the preparation they received.

2. Breath Collections

Expired air was sampled for H₂ concentration as described in Suarez et al., New Engl J Med 333:1-4 (1995).

B. Analyses

Each breath collection for H₂ determination was analyzed for CO₂ (Capstar-100, CWE Inc., Ardmore, Pa.) to insure that an adequate alveolar sample had been collected. The H₂ concentration of the rare sample that contained less than 4.5% CO₂ (5 out of 360 samples) were normalized to 5% CO₂ (observed H₂ concentration) (5%/observed CO₂ concentration). Hydrogen concentration was determined by gas chromatography using a molecular sieve column, nitrogen as the carrier gas, and a reduction detector (Trace Analytical, Menlo Park, Calif.).

Statistics and calculation. The significance of differences between means observed with the two treatments was determined by paired, two-tailed t-test. The quantity of carbohydrate malabsorption induced by the tea preparation was estimated by first determining the difference between the sum of breath H₂ concentrations observed over hours 1-8 when subjects ingested tea versus placebo. The g of carbohydrate represented by this H₂ difference was then compared to the previously observed difference in the sum H₂ of concentrations over hours 1-8 when 55 healthy subjects ingested 10 g of lactulose or a non-caloric beverage. (Strocchi et al., Gastroenterol 105:1404-10 (1993). The excess sum of breath H₂ concentrations observed for 10 g of lactulose averaged 6.2 μmol/L; and carbohydrate malabsorption induced by tea was estimated from the formula:
Malabsorption (g)=(Σ[H2] hours 1-8tea−Σ[H2] hours 1-8placebo) (10 g/6.2 μmol/L) (eq 1)
C. Results

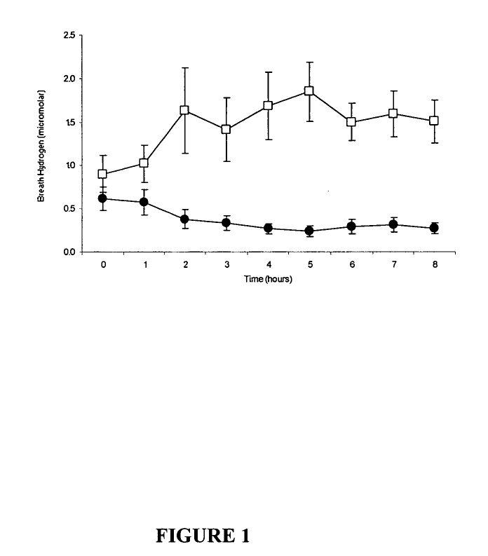

Breath H₂ concentration. The hourly H₂ concentrations (mean±sem) observed following ingestion of the rice and butter meal with each of the two treatments are shown in FIG. 1. Significance of differences was determined by paired, two-tailed t-test. Values obtained with the two treatments were not significantly different for zero and 1 hour measurements. Each hourly measurement for hours 2-8 was significantly greater when the tea extract combination was ingested (p=0.026 at hour 2, p=0.013 at 3 hours, and p<0.003 for hours 4-8).

The H₂ concentrations were not significantly different at baseline and 1 hour. However, the curves significantly diverged by 2 hours, with the breath H₂ concentration being significantly greater in the group receiving tea extract combination at each hourly time point from 2 through 8 hours. The sum of the breath H₂ concentrations for hours 1-8 (a value that closely approximates the area under the curve for 1-8 hours) averaged 12.2±2.0 μmol/L and 2.7±0.6 μmol/L in the groups receiving tea and placebo, respectively (p<0.001). Using eq 1, this H₂ difference (9.5 μmol/L) indicates that tea approximately 15 g of the 60 g of carbohydrate in the meal was not absorbed over the 8 hour test period.

Symptoms. Table 1 shows a comparison of symptoms reported by healthy volunteers in Study 1 for the eight hour period following ingestion of a standard carbohydrate- and lipid-containing meal with the tea extract combination of green tea, fermented tea and mulberry tea, or placebo. In a crossover design, 20 subjects were studied after eating a standard meal ingested with tea extract combination or a placebo. Symptoms were rated on a linear scale of 0 (none) to 4 (severe), and data represent mean±sem. P values were calculated from two-tailed, paired t-tests, not corrected for multiple comparisons. No significant difference (p<0.05) was observed for any symptom on the day of tea extract combination ingestion versus that of the placebo day. Similarly, no significant differences in symptoms were observed between the two treatments in Study 2 (data not shown).

TABLE 1

| Symptom | Combination | Placebo | p-value |
|---|---|---|---|
| Headache | 1.16 ± 0.27 | 0.71 ± 0.27 | 0.11 |
| Fullness | 0.77 ± 0.17 | 0.59 ± 0.19 | 0.44 |
| Itching | 0.07 ± 0.05 | 0.02 ± 0.02 | 0.33 |
| Incomplete evacuation | 0.23 ± 0.14 | 0.13 ± 0.10 | 0.33 |
| Nausea | 0.70 ± 0.23 | 0.23 ± 0.19 | 0.06 |
| Excessive rectal gas | 0.61 ± 0.21 | 0.21 ± 0.12 | 0.12 |
| Fatigue | 1.13 ± 0.25 | 0.97 ± 0.26 | 0.56 |
| Bloating | 0.45 ± 0.19 | 0.26 ± 0.13 | 0.31 |
| Abdominal pain | 0.41 ± 0.20 | 0.13 ± 0.17 | 0.67 |

Subjects ingested standard meals with the tea extract combination or a placebo beverage. The initial test meal contained 60 g of carbohydrate (50 g of starch as white rice, 10 g of sucrose in the tea) and 10.2 g of fat . White rice was used as the complex carbohydrate since, in contrast to most complex carbohydrates, rice starch is nearly completely absorbed by healthy subjects. Thus, a rice meal allows breath testing to more sensitively determine if a manipulation significantly increases H₂ excretion, i.e., causes starch malabsorption. As shown in FIG. 1, breath H₂ concentration declined with the placebo indicating that residual fermentable colonic substrate was not replenished via malabsorption of carbohydrate in the test meal. In contrast, the tea extract combination resulted in increased breath H₂, with measurements for tea versus placebo showing significant differences for each hourly measurement between 2-8 hours. Thus, the tea extract combination clearly induced malabsorption of the starch and/or sucrose.

Tea extract combination-induced carbohydrate malabsorption was estimated by comparing the difference in breath H₂ concentration with the combination versus placebo to the H₂ concentrations observed previously in healthy volunteers ingesting 10 of lactulose (see, eq (1)). This calculation suggested that about 15 g of the 60 g of carbohydrate in the test meal was not absorbed. This may be a minimal estimate since non-absorbed material in the test meal could have been fermented less rapidly than lactulose. (Christl et al., Quantitative measurement of hydrogen and methane from fermentation using a whole body calorimeter. Gastroenterol 102:1269-77 (1992).

The ability of the tea extract combination to inhibit carbohydrate absorption has potential clinical utility for weight control and treatment of diabetes. Assuming that the combination causes malabsorption of 25% of ingested carbohydrate, striking weight loss would be expected providing that caloric intake was not commensurately increased and the caloric content of malabsorbed carbohydrate was unavailable to the host. Malabsorption of 25% of 400 g of carbohydrate per day would reduce caloric availability by roughly 146,000 calories (16 kilograms of fat) per year. While it is commonly assumed that the host obtains no calories from materials entering the colon, the colonic absorption of carbohydrate fermentation products results in an appreciable conservation of calories. (Bond et al., Fate of soluble carbohydrate in the colon of rats and man. J Clin Invest 57:1158-64 (1976). Thus, weight loss would be less than the predicted 16 kg/year.

For centuries, teas have been used as a treatment for diabetes mellitus in Asia. Multiple studies have demonstrated that extracts of mulberry and other teas reduce blood glucose in type-2 diabetics and in animal models of diabetes. This hypoglycemic effect generally has been attributed to alterations of the intermediary metabolism of glucose. The present study indicates that tea combination-induced carbohydrate malabsorption also could influence blood glucose concentrations.

### V. References

1. Pereira M A, Swain J, Goldfine A B, Rifai N, Ludwig D S. Effects of a low-glycemic load diet on resting energy expenditure and heart disease risk factors during weight loss. JAMA 2004; 292(20):2482-90
2. Foster G D, Wyatt H R, Hill J O, McGuckin B G, Brill C, Mohammed B S, Szapary P O, Rader D J, Edman J S, and Klein S. A randomized trial of a low-carbohydrate diet for obesity. N. Engl. J. Med. 2003; 348(21):2082-2090
3. Pencek R R, Koyama Y, Lacy D B, James F D, Fueger P T, Jabbour K, Williams P E and Wasserman D H. Transporter-mediated absorption is the primary route of entry and is required for passive absorption of intestinal glucose into the blood of conscious dogs. J. Nutr. 2002; 132:1929-1934
4. Marshall J J, Lauda C M. Purification and properties of phaseolamin, an inhibitor of alpha-amylase, from the kidney bean, Phaseolus vulgaris. J Biol Chem. 1975;250 (20):8030-7
5. O'Donnell M D and McGeeney K F. Purification and properties of an alpha-amylase inhibitor from wheat. Biochimica et Biophysica Acta 1976; 422:159-169
6. Layer P, Carlson G L and DiMagno E P. Partially purified white bean amylase inhibitor reduces starch digestion in vitro and inactivates intraduodenal amylase in humans. Gastroenterology 1985; 88:1895-902
7. Layer P, Zinsmeister A R and DiMagno E P. Effects of decreasing intraluminal amylase activity on starch digestion and postprandial gastrointestinal function in humans. Gastroenterology 1986; 91:41-8
8. Editorial. The starch-blocker idea. Lancet 1983; 8324:569-70
9. Bo-Linn G W. Starch blockers-their effect on calorie absorption from a high-starch meal. N. Engl J Med. 1982; 307:1413-6
10. Carlson G L, Li BUK, Bass P and Olsen W A. A bean alpha-amylase inhibitor formulation (starch blocker) is ineffective in man. Science 1983; 219:393-395
11. Bischoff H. The mechanism of alpha-glucosidase inhibition in the management of diabetes. Clin Invest Med. 1995; 18:303-11
12. Rachman J, Turner R C. Drugs on the horizon for treatment of type 2 diabetes. Diabet Med. 1995; 12(6):467-78.
13. Scheen A J. Is there a role for alpha-glucosidase inhibitors in the prevention of type 2 diabetes mellitus? Drugs 2003; 63:933-951
14. Kobayashi Y, Suzuki M, Satsu H, Arai S, Hara Y, Suzuki K, Miyamoto Y and Shimizu M. Green tea polyphenols inhibit the sodium dependent glucose transporter of intestinal epithelial cells by a competitive mechanism. J. Agric. Food Chem 2000; 48:5618-5623
15. Murao et al. Agric. Biol. Chem. 1981; 45:2599-2604
16. Asano N, Oseki K, Tomioka E, Kizu H, and Matsui K. N-containing sugars from Morus alba and their glycosidase inhibitory activities. Carbohydr. Res. 1994; 259:243-55
17. Han L-K, Kimura Y, Kawashima M, Takaku T, Taniyama T, Hayashi T, Zheng Y-N and Okuda H. Anti-obesity effects in rodents of dietary teasaponin, a lipase inhibitor. International J. Of Obesity. 2001; 25:1459-1464
18. Nakai M, Fukui Y, Asami S, Toyoda-Ono Y, Iwashita T, Shibata H, Mitsunaga T, Hashimoto F, Kiso Y. Inhibitory effects of Oolong tea polyphenols on pancreatic lipase in vitro. J. Agric Food Chem. 2005; 53:4593-4598
19. Juhel C, Armand M., Pafumi Y., Rosier C, Vandermander J, Lairon D. Green tea extract (AR25) inhibits lipolysis of triglycerides in gastric and duodenal medium in vitro. J. Nutr Biochem. 2000; 11:45-51
20. Ikeda I, Tsuda K, Suzuki Y, Kobayashi M, Unno T, Tomoyori H, Goto H, Kawata Y, Imaizumi K, Nozawa A, Kakuda T. Tea catechins with a galloyl moiety suppress postprandial hypertriacylglycerolemia by delaying lymphatic transport of dietary fat in rats. J. Nutr. 2005; 135:155-9
21. Astrup A. The role of dietary fat in obesity. Semin. Vasc. Med. 2005; 5(1): 40-7
22. Zhang J. and Kashket S. Inhibition of salivary amylase by black and green teas and their effects on the intraoral hydrolysis of starch. Caries Res. 1998; 32(3): 233-8
23. Ikea I, Imasato Y, Sasaki E. Tea catechins decrease micellar solubility and intestinal absorption of cholesterol in rats. Biochim. Biophys Acta. 1992; 1127:141-146
24. Muramatsu K, Fukuyo M, Hara Y. Effect of green tea catechins on plasma cholesterol level in cholesterol-fed rats. J. Nutr. Sci. Vitaminol. 1986: 613-622
25. Chan P T, Fong W P, Cheung Y L, Huang Y. Ho W K, Chen Z Y. Jasmine green tea epicatechins are hypolipidemic in hamsters (Mesocricetus auratus) fed a high fat diet. J. Nutr. 1999: 129:1094-1101

We claim:

1. A composition comprising a pancreatic lipase inhibitor, an alpha glucosidase inhibitor, and a sodium dependent glucose transporter inhibitor.

2. The composition of claim 1, wherein said pancreatic lipase inhibitor comprises polymerized catechin, fermented tea extract, or a combination thereof

3. The composition of claim 2, wherein said polymerized catechin comprises theaflavin.

4. The composition of claim 2, wherein said fermented tea extract is black tea extract.

5. The composition of claim 1, wherein said alpha glucosidase inhibitor comprises 1-deoxynojirimycin, mulberry leaf extract, or a combination thereof

6. The composition of claim 1, wherein said sodium dependent glucose transporter inhibitor comprises epicatechin gallate, green tea extract, or a combination thereof.

7. The composition of claim 1, said pancreatic lipase inhibitor is a polymerized catechin or a fermented tea extract; said alpha glucosidase inhibitor is a mulberry leaf extract, and said sodium dependent glucose transporter inhibitor is green tea extract.

8. The composition of claim 7, wherein said polymerized catechin is theaflavin.

9. The composition of claim 7, wherein said fermented tea extract is black tea extract.

10. A method for controlling glucose and lipid uptake in an individual comprising administering to the individual a composition comprising a pancreatic lipase inhibitor, an alpha glucosidase inhibitor, and a sodium dependent glucose transporter inhibitor, thereby controlling glucose and lipid uptake.

11. The method of claim 10, comprising administering said pancreatic lipase inhibitor, said alpha glucosidase inhibitor and said sodium dependent glucose transporter inhibitor simultaneously or at different times.

12. The method of claim 10, wherein said pancreatic lipase inhibitor, said alpha glucosidase inhibitor and said sodium dependent glucose transporter inhibitor are formulated in a single formulation.

13. The method of claim 10, wherein said pancreatic lipase inhibitor, said alpha glucosidase inhibitor and said sodium dependent glucose transporter inhibitor are administered orally.

14. The method of claim 10, wherein the individual has diabetes or is at risk of diabetes.

15. The method of claim 10, wherein the individual is obese or is at risk of obesity.

16. The method of claim 10, wherein said pancreatic lipase inhibitor, said alpha glucosidase inhibitor and said sodium dependent glucose transporter inhibitor are administered before a meal or with a meal.

17. The method of claim 10, wherein said pancreatic lipase inhibitor comprises theaflavin or a fermented tea extract, said alpha glucosidase inhibitor comprises a mulberry leaf extract, and said sodium dependent glucose transporter inhibitor comprises a green tea extract.

18. A kit comprising the composition of claim 1, and optionally comprising an instruction for using the composition for controlling glucose and lipid uptake in an individual.

19. A method for inducing carbohydrate malabsorption, comprising administering to an individual a composition comprising a pancreatic lipase inhibitor, an alpha glucosidase inhibitor, and a sodium dependent glucose transporter inhibitor, thereby inducing carbohydrate malabsorption.

20. The method of claim 19, wherein said pancreatic lipase inhibitor comprises theaflavin or a fermented tea extract, said alpha glucosidase inhibitor comprises a mulberry leaf extract, and said sodium dependent glucose transporter inhibitor comprises a green tea extract.
